(19) United States
(12) Patent Application Publication
(10) Pub. No.: US 2025/0263360 A1
(43) Pub. Date: Aug. 21, 2025

(54) METHOD FOR PRODUCING POLYFLUORINATED TERTIARY ALCOHOLS

(71) Applicant: Innolith Technology AG, Basel (CH)

(72) Inventors: Michael Hassler, Bruchsal (DE); Lukas Gooßen, Hattingen (DE); Gregor Heinrich, Bochum (DE); Mykhailo Kondratiuk, Essen (DE)

(21) Appl. No.: 19/190,078

(22) Filed: Apr. 25, 2025

Related U.S. Application Data
(63) Continuation of application No. PCT/EP2023/079477, filed on Oct. 23, 2023.

(30) Foreign Application Priority Data
Oct. 28, 2022 (DE) 10 2022 128 696.7

(51) Int. Cl. C07C 31/38; C07C 29/143

(52) U.S. Cl. CPC C07C 31/38; C07C 29/143

(57) ABSTRACT

A method is disclosed for producing polyfluorinated alcohols of formula (I)

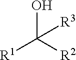

starting from a ketone of formula (II),

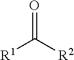

and a carboxylic acid salt of formula (III) (R³COO)ₓY. The substituents R¹ and R² can be selected from C₁-C₁₀ alkyl, C₃-C₁₀ cycloalkyl, C₆-C₁₄ aryl, and C₅-C₁₄ heteroaryl. The substituents may be unsubstituted or partially or completely fluorinated. R³ is a partially or completely fluorinated C₁-C₁₀ alkyl, Y is a cation of K, Li, Na, Cs, Mg, Ca, Fe, Cu, Ag, and Zn, and x is 1 or 2. In the method, the R³ group of the carboxylic acid salt is transferred to the carbonyl carbon of the ketone of formula (II) with the release of CO₂.

### RELATED APPLICATIONS

This application is a continuation of PCT/EP2023/079477, filed Oct. 23, 2023, which claims priority to EP 10 2022 128 696.7, filed Oct. 28, 2022, the entire disclosures of both of which are hereby incorporated herein by reference.

### BACKGROUND

This disclosure relates to a method for producing polyfluorinated tertiary alcohols. It also relates to the use of polyfluorinated alcohols produced according to the method described above.

Polyfluorinated alcohols serve as starting materials for the synthesis of chemical compounds used in numerous consumer products. However, no efficient, one-step synthesis for producing polyfluorinated tertiary alcohols is known in the prior art. U.S. Pat. No. 3,317,616 A, by contrast, discloses a stepwise synthesis of polyfluorinated tertiary alcohols starting from polyfluoroalkyl ketones.

A common approach to producing polyfluorinated alcohols is based on the addition of Me₃SiCF₃ (Ruppert-Prakash reagent). The addition of aldehydes, as described in R. Filler, R. M. Schure, J. Org. Chem. 1967, 32, 1217-1219, yields secondary alcohol, whereas the addition of ketones, as reported in G. K. S. Prakash, M. M andal, Journal of Fluorine Chemistry 2001, 112, 123-131, results in tertiary alcohols.

However, this synthesis strategy, consisting of the trifluoromethylation of ketones and aldehydes with preformed polylfluoroalkyl nucleophiles such as Me₃SiCF₃ (Ruppert-Prakash reagent), is economically disadvantageous due to the high costs of such reagents.

Chang et al., in the Journal of Fluorine Chemistry 2005, 126 (6), 937-940 and Tetrahedron Letters 2005, 46, 3161-3164, reported a method that eliminates the need for Ruppert-Prakash reagents. This method involves decarboxylative polylfluoroalkylation, where the polylfluoroalkyl nucleophile is generated in situ via decarboxylation from polyfluorocarboxylate salts and added to aldehydes and ketones.

According to this method, benzaldehyde can be reacted with trifluoroacetates to form the corresponding alcohol in the presence of a stoichiometric quantity of a Cu(I) halide:

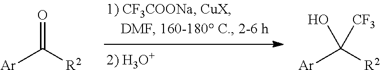

Starting from benzaldehyde (R²═H), the corresponding secondary alcohol was produced with a very high yield of 99%. However, only traces of the desired product, the tertiary alcohol, were detected in attempted conversions of ketones (R²═CH₃) and esters (R²═OCH₃).

Gooßen et al. reported in the European Journal 2015, 21, 17220-17223 and the Journal of Fluorine Chemistry 2017, 198, 89-93, that the presence of iron catalysts and the use of DMF as a solvent favor decarboxylating perfluoroalkylations, whereby only reactions with aldehydes that yielded secondary alcohols were disclosed in the above-mentioned documents. The achieved yield was approximately 60%:

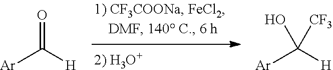

### SUMMARY AND DESCRIPTION

In view of the aforementioned disadvantages of the known syntheses and the high demand for polyfluorinated tertiary alcohols, the purpose of this disclosure was therefore to teach a new, efficient, and cost-effective synthesis for producing polyfluorinated tertiary alcohols.

Thus, a method for producing polyfluorinated alcohols of formula (I) is provided,

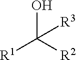

wherein
R¹ is selected from the group consisting of C₁-C₁₀ alkyl, C₃-C₁₀ cycloalkyl, C₆-C₁₄ aryl, and C₅-C₁₄ heteroaryl, wherein the substituents mentioned may be unsubstituted or partially or completely fluorinated;
R² is selected from the group consisting of C₁-C₁₀ alkyl, C₃-C₁₀ cycloalkyl, C₆-C₁₄ aryl, and C₅-C₁₄ heteroaryl, wherein the substituents mentioned may be unsubstituted or partially or completely fluorinated;
R³ is a partially or completely fluorinated C₁-C₁₀ alkyl;
starting from a ketone of formula (II),

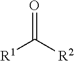

wherein
R¹ is selected from the group consisting of C₁-C₁₀ alkyl, C₃-C₁₀ cycloalkyl, C₆-C₁₄ aryl, and C₅-C₁₄ heteroaryl, wherein the substituents mentioned may be unsubstituted or partially or completely fluorinated;
R² is selected from the group consisting of C₁-C₁₀ alkyl, C₃-C₁₀ cycloalkyl, C₆-C₁₄ aryl, and C₅-C₁₄ heteroaryl, wherein the substituents mentioned may be unsubstituted or partially or completely fluorinated,
and a carboxylic acid salt of formula (III) (R³COO)ₓY, wherein
R³ is a partially or completely fluorinated C₁-C₁₀ alkyl;
Y is a cation selected from the group consisting of K, Li, Na, Cs, Mg, Ca, Fe, Cu, Ag, and Zn;
X is 1 or 2;
is characterized in that
the R³ group of the carboxylic acid salt is transferred to the carbonyl carbon of the ketone of formula (II) with the release of CO₂.

The method according to this disclosure is highly efficient and cost-effective due to the single-step synthesis that eliminates the need for expensive reagents. It enables the production of polyfluorinated tertiary alcohols on a large scale.

In the context of this disclosure, the term “polyfluorinated alcohols” refers to alcohols of formula (I) in which at least two hydrogen atoms on at least one carbon atom of the substituent R¹, R², or R³ have been replaced by fluorine atoms.

In the context of this disclosure, the term “C₁-C₁₀ alkyl” refers to linear or branched saturated hydrocarbon groups having one to ten carbon atoms. These include, in particular, methyl, ethyl, n-propyl, isopropyl, n-butyl, sec-butyl, isobutyl, tert-butyl, n-pentyl, isopentyl, 2,2-dimethylpropyl, n-hexyl, isohexyl, 2-ethylhexyl, n-heptyl, isoheptyl, n-octyl, isooctyl, n-nonyl, n-decyl and the like.

In the context of this disclosure, the term “C₂-C₁₀ alkenyl” refers to unsaturated linear or branched hydrocarbon groups having two to ten carbon atoms, wherein the hydrocarbon groups contain at least one C—C double bond. These include in particular ethenyl, 1-propenyl, 2-propenyl, 1-n-butenyl, 2-n-butenyl, isobutenyl, 1-pentenyl, 1-hexenyl, 1-heptenyl, 1-octenyl, 1-nonenyl, 1-decenyl, and the like.

In the context of this disclosure, the term “C₃-C₁₀ cycloalkyl” refers to cyclic, saturated hydrocarbon groups having three to ten carbon atoms. These include, in particular, cyclopropyl, cyclobutyl, cyclopentyl, cyclohexyl, cycloheptyl, cyclohexyl, cyclononyl, and cyclodecanyl.

In the context of this disclosure, the term “C₆-C₁₄ aryl” refers to aromatic hydrocarbon groups having six to fourteen carbon atoms in the ring. These include in particular phenyl (C₆H₅ group), naphthyl (C₁₀H₇ group) and anthracyl (C₁₄H₉ group).

In the context of this disclosure, the term “C₅-C₁₄ heteroaryl” refers to aromatic hydrocarbon groups with five to fourteen ring hydrocarbon atoms in which at least one hydrocarbon atom is replaced or exchanged by a nitrogen, oxygen or sulfur atom. These include in particular pyrrolyl, furanyl, thiophenyl, pyrridinyl, pyranyl, thiopyranyl and the like. All of the aforementioned hydrocarbon groups are bonded to the central atom according to formula (I) via an oxygen atom, respectively.

An advantageous development of the method according to this disclosure involves carrying out the reaction in a solvent selected from the group consisting of DMF, NMP, DMAc, and DMSO.

The aforementioned solvent is suitable for stabilizing the fluorinated carbanion during the reaction, although this disclosure is not limited to this.

In the context of this disclosure, the term “fluorinated carbanion” refers to a negatively charged carbon atom whose hydrogen atoms have been replaced by fluorine atoms.

In the context of this disclosure, the abbreviations DMF, NMP, DM Ac, and DMSO refer to the following:
DMF—N,N-dimethylformamide
NMP—N-methyl-2-pyrrolidone
DMAc—N,N-dimethylacetamide
DMSO—dimethyl sulfoxide

A further advantageous development of the method according to this disclosure provides for the reaction to be carried out in DMF or DMSO as a solvent.

A most preferred development of the method according to this disclosure provides for the reaction to be carried out in DMF.

The reaction temperature in the method according to this disclosure preferably ranges from 100° C. to 150° C., more preferably from 130° C. to 150° C., and most preferably from 135° C. to 145° C.

A most preferred development of the method according to this disclosure provides for the reaction temperature to be 140° C.

A further advantageous development of the method according to this disclosure provides for the reaction to be carried out under anhydrous conditions.

In the method according to this disclosure, this prevents the very sensitive polyfluorinated ketones, as reactants in the reaction, from converting to the corresponding hydrates in the presence of traces of water, which thermally decompose to form carbonic acids, and thus have no negative influence on the yield of the desired tertiary polyfluorinated alcohols. Furthermore, this also prevents the CF₃ anion from being directly converted to HCF₃ due to its very high pK_{S} value, thereby preventing the reaction from proceeding in the presence of strong bases.

A further advantageous development of the method according to this disclosure provides for the reaction to be carried out in the presence of a catalyst.

In the case of less reactive reactants, this embodiment of this disclosure has proven especially beneficial for achieving higher yields.

In the context of this disclosure, all catalysts known in the prior art and suitable for this purpose, particularly Lewis acids, can be used as catalysts for the reaction.

A further advantageous development of the method according to this disclosure provides for the reaction to be carried out in the presence of an iron catalyst.

In the method according to this disclosure, the iron catalyst is preferably selected from the group consisting of FeCl₂, FeCl₃, FeBr₃, FeF₃, FeTFA₃, FeSO₄, Fe₂(SO₄)₃, and more preferably from FeCl₂, FeCl₃. FeCl₃ is most preferred as a catalyst.

If FeCl₃ is used as a catalyst, bpy (2,2′-Bipyridine), TMEDA (N,N,N′,N′-tetramethylethylenediamine), or [2.2.2]cryptand can be added as ligands to the reaction in a 1:1 ratio.

In the method according to this disclosure, the quantity of iron catalysts preferably ranges from 10 to 75 mol %, more preferably 15 to 45 mol %.

A most preferred development of the method according to this disclosure provides for the quantity of iron catalyst to be 20 to 30 mol %.

A further advantageous development of the method according to this disclosure provides for the catalyst to be selected from the group consisting of KOtBu, K₂CO₃, KCl, ZnCl₂, CoCl₂, MnCl₂, InCl₃, GaBr₃, CuI, CuBr, AgBF₃, and Sc(OTf)₃.

In the method according to this disclosure, the quantity of catalyst preferably ranges from 10 to 75 mol %, more preferably 10 to 30 mol %.

In the case of a gaseous reactant, the method according to this disclosure can be carried out using one of the following methods, but is not limited to them: crimped cap vials, gas burettes, and autoclaves. If an autoclave is used, the reaction pressure is 0.5 to 6.5 bar, preferably 2.0 to 4.0 bar, and most preferably 3.0 bar.

The reaction time ranges from 1 to 24 hours.

The method according to this disclosure is suitable for producing polyfluorinated alcohols of formula (I),

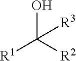

wherein
R¹ is a C₁-C₁₀ alkyl, which may be unsubstituted or partially or completely fluorinated;
R² is a C₁-C₁₀ alkyl, which may be unsubstituted or partially or completely fluorinated;
R³ is a partially or completely fluorinated C₁-C₁₀ alkyl.

The method according to this disclosure is well-suited for producing polyfluorinated alcohols of formula (I),

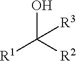

wherein
R¹ is a C₁-C₁₀ alkyl, which may be partially or completely fluorinated;
R² is a C₁-C₁₀ alkyl, which may be partially or completely fluorinated;
R³ is a partially or completely fluorinated C₁-C₁₀ alkyl.

The method according to this disclosure is especially well-suited for producing polyfluorinated alcohols of formula (I),

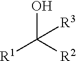

wherein
R¹ is a C₁-C₁₀ alkyl, which is completely fluorinated;
R² is a C₁-C₁₀ alkyl, which is completely fluorinated;
R³ is a completely fluorinated C₁-C₁₀ alkyl.

The method according to this disclosure is most suited for producing polyfluorinated alcohols of formula (I),

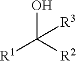

wherein
R¹ is selected from the group consisting of CF₃, CF₂CF₃, CF(CF₃)₂, C(CF₃) 3, or CF₂CF₂CF₃;
R² is selected from the group consisting of CF₃, CF₂CF₃, CF(CF₃) 2, C(CF₃) 3, or CF₂CF₂CF₃;
R³ is selected from the group consisting of CF₃, CF₂CF₃, CF(CF₃)₂, C(CF₃) 3, or CF₂CF₂CF₃.

The method according to this disclosure is most suited for producing polyfluorinated alcohols:
I-1a to 1-156a, wherein R³ is CF₃ and the respective combination of R¹ and R² are listed in Table 1;
1-1b to 1-156b, wherein R³ is CF₂CF₃ and the respective combination of R¹ and R² are listed in Table 1;
I-1c to 1-156c, wherein R³ is CF(CF₃) 2 and the respective combination of R 1 and R² are listed in Table 1;
1-1d to 1-156d, wherein R³ is C(CF₃) 3 and the respective combination of R¹ and R² are listed in Table 1;
I-1e to 1-156e, wherein R³ is CF₂CF₂CF₃ and the respective combination of R¹ and R² are listed in Table 1.

TABLE 1

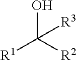

| No. | R¹ | R² |
|---|---|---|
| 1-1 | CH₃ | CH₃ |
| 1-2 | CH₂F | CH₃ |
| 1-3 | CHF₂ | CH₃ |
| 1-4 | CF₃ | CH₃ |
| 1-5 | CH₂CH₃ | CH₃ |
| 1-6 | CH₂CH₂F | CH₃ |
| 1-7 | CH₂CHF₂ | CH₃ |
| 1-8 | CH₂CF₃ | CH₃ |
| 1-9 | CF₂CF₃ | CH₃ |
| 1-10 | CF(CF₃)₂ | CH₃ |
| 1-11 | C(CF₃)₃ | CH₃ |
| 1-12 | CF₂CF₂CF₃ | CH₃ |
| 1-13 | CH₃ | CH₂F |
| 1-14 | CH₂F | CH₂F |
| 1-15 | CHF₂ | CH₂F |
| 1-16 | CF₃ | CH₂F |
| 1-17 | CH₂CH₃ | CH₂F |
| 1-18 | CH₂CH₂F | CH₂F |
| 1-19 | CH₂CHF₂ | CH₂F |
| 1-20 | CH₂CF₃ | CH₂F |
| 1-21 | CF₂CF₃ | CH₂F |
| 1-22 | CF(CF₃)₂ | CH₂F |
| 1-23 | C(CF₃)₃ | CH₂F |
| 1-24 | CF₂CF₂CF₃ | CH₂F |
| 1-25 | CH₃ | CHF₂ |
| 1-26 | CH₂F | CHF₂ |
| 1-27 | CHF₂ | CHF₂ |
| 1-28 | CF₃ | CHF₂ |
| 1-29 | CH₂CH₃ | CHF₂ |
| 1-30 | CH₂CH₂F | CHF₂ |
| 1-31 | CH₂CHF₂ | CHF₂ |
| 1-32 | CH₂CF₃ | CHF₂ |
| 1-33 | CF₂CF₃ | CHF₂ |
| 1-34 | CF(CF₃)₂ | CHF₂ |
| 1-35 | C(CF₃)₃ | CHF₂ |
| 1-36 | CF₂CF₂CF₃ | CHF₂ |
| 1-37 | CH₃ | CHF₂ |
| 1-38 | CH₂F | CHF₂ |
| 1-39 | CHF₂ | CHF₂ |
| 1-40 | CF₃ | CHF₂ |
| 1-41 | CH₂CH₃ | CHF₂ |
| 1-42 | CH₂CH₂F | CHF₂ |
| 1-43 | CH₂CHF₂ | CHF₂ |
| 1-44 | CH₂CF₃ | CHF₂ |
| 1-45 | CF₂CF₃ | CHF₂ |
| 1-46 | CF(CF₃)₂ | CHF₂ |
| 1-47 | C(CF₃)₃ | CHF₂ |
| 1-48 | CF₂CF₂CF₃ | CHF₂ |
| 1-49 | CH₃ | CH₂CH₃ |
| 1-50 | CH₂F | CH₂CH₃ |
| 1-51 | CHF₂ | CH₂CH₃ |
| 1-52 | CF₃ | CH₂CH₃ |
| 1-53 | CH₂CH₃ | CH₂CH₃ |
| 1-54 | CH₂CH₂F | CH₂CH₃ |
| 1-55 | CH₂CHF₂ | CH₂CH₃ |
| 1-56 | CH₂CF₃ | CH₂CH₃ |
| 1-57 | CF₂CF₃ | CH₂CH₃ |
| 1-58 | CF(CF₃)₂ | CH₂CH₃ |
| 1-59 | C(CF₃)₃ | CH₂CH₃ |
| 1-60 | CF₂CF₂CF₃ | CH₂CH₃ |
| 1-61 | CH₃ | CH₂CH₂F |
| 1-62 | CH₂F | CH₂CH₂F |
| 1-63 | CHF₂ | CH₂CH₂F |
| 1-64 | CF₃ | CH₂CH₂F |
| 1-65 | CH₂CH₃ | CH₂CH₂F |
| 1-66 | CH₂CH₂F | CH₂CH₂F |
| 1-67 | CH₂CHF₂ | CH₂CH₂F |
| 1-68 | CH₂CF₃ | CH₂CH₂F |
| 1-69 | CF₂CF₃ | CH₂CH₂F |
| 1-70 | CF(CF₃)₂ | CH₂CH₂F |
| 1-71 | C(CF₃)₃ | CH₂CH₂F |
| 1-72 | CF₂CF₂CF₃ | CH₂CH₂F |
| 1-73 | CH₃ | CH₂CHF₂ |
| 1-74 | CH₂F | CH₂CHF₂ |
| 1-75 | CHF₂ | CH₂CHF₂ |
| 1-76 | CF₃ | CH₂CHF₂ |
| 1-77 | CH₂CH₃ | CH₂CHF₂ |
| 1-78 | CH₂CH₂F | CH₂CHF₂ |
| 1-79 | CH₂CHF₂ | CH₂CHF₂ |
| 1-80 | CH₂CF₃ | CH₂CHF₂ |
| 1-81 | CF₂CF₃ | CH₂CHF₂ |
| 1-82 | CF(CF₃)₂ | CH₂CHF₂ |
| 1-83 | C(CF₃)₃ | CH₂CHF₂ |
| 1-84 | CF₂CF₂CF₃ | CH₂CHF₂ |
| 1-85 | CH₃ | CH₂CF₃ |
| 1-86 | CH₂F | CH₂CF₃ |
| 1-87 | CHF₂ | CH₂CF₃ |
| 1-88 | CF₃ | CH₂CF₃ |
| 1-89 | CH₂CH₃ | CH₂CF₃ |
| 1-90 | CH₂CH₂F | CH₂CF₃ |
| 1-91 | CH₂CHF₂ | CH₂CF₃ |
| 1-92 | CH₂CF₃ | CH₂CF₃ |
| 1-93 | CF₂CF₃ | CH₂CF₃ |
| 1-94 | CF(CF₃)₂ | CH₂CF₃ |
| 1-95 | C(CF₃)₃ | CH₂CF₃ |
| 1-96 | CF₂CF₂CF₃ | CH₂CF₃ |
| 1-97 | CH₃ | CF₂CF₃ |
| 1-98 | CH₂F | CF₂CF₃ |
| 1-99 | CHF₂ | CF₂CF₃ |
| 1-100 | CF₃ | CF₂CF₃ |
| 1-101 | CH₂CH₃ | CF₂CF₃ |
| 1-102 | CH₂CH₂F | CF₂CF₃ |
| 1-103 | CH₂CHF₂ | CF₂CF₃ |
| 1-104 | CH₂CF₃ | CF₂CF₃ |
| 1-105 | CF₂CF₃ | CF₂CF₃ |
| 1-106 | CF(CF₃)₂ | CF₂CF₃ |
| 1-107 | C(CF₃)₃ | CF₂CF₃ |
| 1-108 | CF₂CF₂CF₃ | CF₂CF₃ |
| 1-109 | CH₃ | CF(CF₃)₂ |
| 1-110 | CH₂F | CF(CF₃)₂ |
| 1-111 | CHF₂ | CF(CF₃)₂ |
| 1-112 | CF₃ | CF(CF₃)₂ |
| 1-113 | CH₂CH₃ | CF(CF₃)₂ |
| 1-114 | CH₂CH₂F | CF(CF₃)₂ |
| 1-115 | CH₂CHF₂ | CF(CF₃)₂ |
| 1-116 | CH₂CF₃ | CF(CF₃)₂ |
| 1-117 | CF₂CF₃ | CF(CF₃)₂ |
| 1-118 | CF(CF₃)₂ | CF(CF₃)₂ |
| 1-119 | C(CF₃)₃ | CF(CF₃)₂ |
| 1-120 | CF₂CF₂CF₃ | CF(CF₃)₂ |
| 1-121 | CH₃ | C(CF₃)₃ |
| 1-122 | CH₂F | C(CF₃)₃ |
| 1-123 | CHF₂ | C(CF₃)₃ |
| 1-124 | CF₃ | C(CF₃)₃ |
| 1-125 | CH₂CH₃ | C(CF₃)₃ |
| 1-126 | CH₂CH₂F | C(CF₃)₃ |
| 1-127 | CH₂CHF₂ | C(CF₃)₃ |
| 1-128 | CH₂CF₃ | C(CF₃)₃ |
| 1-129 | CF₂CF₃ | C(CF₃)₃ |
| 1-130 | CF(CF₃)₂ | C(CF₃)₃ |
| 1-131 | C(CF₃)₃ | C(CF₃)₃ |
| 1-132 | CF₂CF₂CF₃ | C(CF₃)₃ |
| 1-133 | CH₃ | CF₂CF₂CF₃ |
| 1-134 | CH₂F | CF₂CF₂CF₃ |
| 1-135 | CHF₂ | CF₂CF₂CF₃ |
| 1-136 | CF₃ | CF₂CF₂CF₃ |
| 1-137 | CH₂CH₃ | CF₂CF₂CF₃ |
| 1-138 | CH₂CH₂F | CF₂CF₂CF₃ |
| 1-139 | CH₂CHF₂ | CF₂CF₂CF₃ |
| 1-140 | CH₂CF₃ | CF₂CF₂CF₃ |
| 1-141 | CF₂CF₃ | CF₂CF₂CF₃ |
| 1-142 | CF(CF₃)₂ | CF₂CF₂CF₃ |
| 1-143 | C(CF₃)₃ | CF₂CF₂CF₃ |
| 1-144 | CF₂CF₂CF₃ | CF₂CF₂CF₃ |
| 1-145 | CH₃ | Phenyl |
| 1-146 | CH₂F | Phenyl |
| 1-147 | CHF₂ | Phenyl |
| 1-148 | CF₃ | Phenyl |
| 1-149 | CH₂CH₃ | Phenyl |
| 1-150 | CH₂CH₂F | Phenyl |
| 1-151 | CH₂CHF₂ | Phenyl |
| 1-152 | CH₂CF₃ | Phenyl |
| 1-153 | CF₂CF₃ | Phenyl |
| 1-154 | CF(CF₃)₂ | Phenyl |
| 1-155 | C(CF₃)₃ | Phenyl |
| 1-156 | CF₂CF₂CF₃ | Phenyl |

The aforementioned compounds can be used to produce electrolytes for battery cells.

The method according to this disclosure is illustrated by the following examples; however, it is not limited to them:

### EXAMPLE 1: PRODUCTION OF C(CF₃)₃OH WITHOUT USING A CATALYST STARTING FROM HEXAFLUOROACETONE

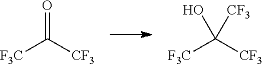 [SMILES: O=C(C(F)(F)F)C(F)(F)F ; OC(C(F)(F)F)(C(F)(F)F)C(F)(F)F]

The production of the aforementioned tertiary alcohol can be carried out according to the following methods:

### Method 1: Reaction Process in Crimped Cap Vials

A kiln-dried 20 ml glass vial with a magnetic stirrer core was filled inside the glovebox with a mixture of pre-dried potassium trifluoroacetate (0.36 mmol, 1.00 equiv.), 30 μl of 1,4-difluorobenzene as an internal NMR standard (0.29 mmol), and 1 ml of dimethylformamide (DMF) before being sealed with a septum cap. In this method, the septum cap is perforated. The excess amount of hexafluoroacetone (approx. 1.1 mmol, approx. 3.00 equiv.) is condensed in the vial, and the reaction is stirred for 16 hours at 140° C. The reaction mixture is then acidified with HCl, and a sample is analyzed using ¹⁹F NMR spectroscopy.

The yield of the desired product is 78%.

### Method 2: Reaction Process in Pressure-Resistant Crimped Cap Vials

A kiln-dried 20 ml glass vial with a magnetic stirrer core was filled inside the glovebox with a mixture of pre-dried potassium trifluoroacetate (0.36 mmol, 1.00 equiv.), 30 μl of 1,4-difluorobenzene as an internal NMR standard (0.29 mmol), and 1 ml of dimethylformamide (DMF) before being sealed with a septum cap. In this method, the septum cap is not perforated. The excess amount of hexafluoroacetone (approx. 1.1 mmol, approx. 3.00 equiv.) is condensed in the vial, and the reaction is stirred for 16 hours at 140° C. The reaction mixture is then acidified with HCl, and a sample is analyzed using ¹⁹F NMR spectroscopy.

The yield of the desired product is 73%.

### Method 3: Reaction Process in Crimped Cap Vials, Dosing of the Substrate Using a Gas Burette

A kiln-dried 20 ml glass vial with a magnetic stirrer core was filled inside the glovebox with a mixture of pre-dried potassium trifluoroacetate (0.6 mmol, 1.00 equiv.), 100 μl of 1,4-difluorobenzene as an internal NMR standard (0.963 mmol), and 1 ml of dimethylformamide (DMF) before being sealed with a septum cap. In this method, the septum cap is not perforated. The excess amount of hexafluoroacetone is measured using a gas burette, condensed in the vial, and the reaction is stirred for 16 hours at 140° C. The reaction mixture is then acidified with HCl, and a sample is analyzed using ¹⁹F NMR spectroscopy.

The yield of the desired product is 76%.

### Method 4: Autoclave

An autoclave (˜70 ml volume) is loaded in the glovebox with potassium trifluoroacetate (2.08 mmol, 1.00 equiv.) and 3.5 ml of dimethylformamide, and equipped with a magnetic stirrer core. After sealing, the excess amount of hexafluoroacetone outside the glovebox—measured using a gas burette—is condensed in the autoclaves and sealed. The reaction is stirred for 16 hours at 140° C. After the reaction is complete, the excess HFA is removed, and the autoclave is opened and flushed. The mixture is then acidified with HCl, and 100 μl of 1,4-difluorobenzene is added as an internal standard. A sample is analyzed using ¹⁹F NMR spectroscopy.

The method was carried out multiple times using different reaction pressures, and the following yields were achieved:

The yield is 58% at a reaction pressure of 0.7 bar.

The yield is 57% at a reaction pressure of 1.1 bar.

The yield is 71% at a reaction pressure of 3.0 bar.

The yield is 57% at a reaction pressure of 6.5 bar.

### EXAMPLE 2: PRODUCTION OF C(CF₃)₃OH USING A CATALYST STARTING FROM HEXAFLUOROACETONE

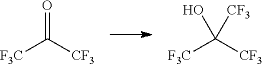 [SMILES: O=C(C(F)(F)F)C(F)(F)F ; OC(C(F)(F)F)(C(F)(F)F)C(F)(F)F]

The production of the aforementioned tertiary alcohol can be carried out according to the following methods:

### Method 1: Reaction Process in a Crimped Cap Vial

A kiln-dried 20 ml glass vial with a magnetic stirrer core was filled inside the glovebox with a mixture of pre-dried potassium trifluoroacetate (0.36 mmol, 1.00 equiv.), iron (III) chloride, 30 μl of 1,4-difluorobenzene as an internal NMR standard (0.29 mmol), and 1 ml of dimethylformamide (DMF) before being sealed with a septum cap. In this method, the septum cap is perforated. The excess amount of hexafluoroacetone (approx. 1.1 mmol, approx. 3.00 equiv.) is condensed in the vial, and the reaction is stirred for 16 hours at 140° C. The reaction mixture is then acidified with HCl, and a sample is analyzed using ¹⁹F NMR spectroscopy.

The method was carried out multiple times using different quantities of FeCl₃, and the following yields were achieved:

The yield is 95% at a quantity of 10 mol % FeCl₃.

The yield is 99% at a quantity of 20 mol % FeCl₃.

The yield is 95% at a quantity of 25 mol % FeCl₃.

The yield is 96% at a quantity of 30 mol % FeCl₃.

The yield is 86% at a quantity of 40 mol % FeCl₃.

### Method 2: Reaction Process in a Crimped Cap Vial, 0.6 Mmol

A kiln-dried 20 ml glass vial with a magnetic stirrer core was filled inside the glovebox with a mixture of pre-dried potassium trifluoroacetate (0.6 mmol, 1.00 equiv.), iron (III) chloride, 30 μl of 1,4-difluorobenzene as an internal NMR standard (0.29 mmol), and 1 ml of dimethylformamide (DMF) before being sealed with a septum cap. In this method, the septum cap is not perforated. The excess amount of hexafluoroacetone (approx. 1.1 mmol, approx. 3.00 equiv.) is condensed in the vial, and the reaction is stirred for 16 hours at 140° C. The reaction mixture is then acidified with HCl, and a sample is analyzed using ¹⁹F NMR spectroscopy.

The method was carried out multiple times using different quantities of FeCl₃, and the following yields were achieved:

The yield is 62% at a quantity of 10 mol % FeCl₃.

The yield is 75% at a quantity of 20 mol % FeCl₃.

The yield is 67% at a quantity of 30 mol % FeCl₃.

The yield is 79% at a quantity of 40 mol % FeCl₃.

### Method 3: Dosing of the Substrate Using a Gas Burette

A kiln-dried 20 ml glass vial with a magnetic stirrer core was filled in the glovebox with a mixture of pre-dried potassium trifluoroacetate (0.6 mmol, 1.00 equiv.), catalyst, 100 μl of 1,4-difluorobenzene as an internal NMR standard (0.963 mmol), and 1 ml of dimethylformamide (DMF) and sealed with a septum cap. In this method, the septum cap is not perforated. The excess amount of hexafluoroacetone is measured using a gas burette, condensed in the vial, and the reaction is stirred for 16 hours at 140° C. The reaction mixture is then acidified with HCl, and a sample is analyzed using ¹⁹F NMR spectroscopy.

The method was carried out multiple times using different catalysts, and the following yields were achieved:

The yield is 77% at a quantity of 10 mol % K₂CO₃.

The yield is 56% at a quantity of 10 mol % KOtBu.

### EXAMPLE 3: PRODUCTION OF PHC(CF₃)₂OH USING A CATALYST STARTING FROM TRIFLUOROACETOPHENONE

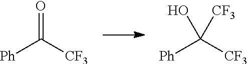 [SMILES: O=C(c1ccccc1)C(F)(F)F ; OC(c1ccccc1)(C(F)(F)F)C(F)(F)F]

A kiln-dried 20 ml glass vial with a magnetic stirrer core was stirred in the glovebox with a mixture of trifluoroacetophenone (0.30 mmol, 1.00 equiv.) with potassium trifluoroacetate (0.36 mmol, 1.20 equiv.), catalyst, and 30 μl of 1,4-difluorobenzene as an internal NMR standard (0.29 mmol, 0.963 equiv.) for 12 hours at 140° C. in 1 ml of dimethylformamide and then acidified with HCl.

The method was carried out multiple times using different catalysts, and the following yields were achieved:

The yield is 53% at a quantity of 30 mol % FeCl₂.

The yield is 63% at a quantity of 30 mol % FeBr₃.

The yield is 63% at a quantity of 30 mol % GaBr₃.

The method was carried out multiple times using different quantities of FeCl₃, and the following yields were achieved:

The yield is 45% at a quantity of 15 mol % FeCl₃.

The yield is 81% at a quantity of 30 mol % FeCl₃.

The yield is 85% at a quantity of 45 mol % FeCl₃.

The yield is 73% at a quantity of 60 mol % FeCl₃.

The yield is 71% at a quantity of 75 mol % FeCl₃.

The method was carried out with 30 mol % of FeCl₃, whereby different ligands were used:

The yield is 77% at a quantity of 30 mol % FeCl₃ and bpy as a ligand.

The yield is 89% at a quantity of 30 mol % FeCl₃ and TMEDA as a ligand.

The yield is 42% at a quantity of 30 mol % FeCl₃ and [2.2.2]cryptand as a ligand.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method for producing polyfluorinated alcohols of formula (I) 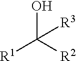
wherein
R¹ is selected from the group consisting of C₁-C₁₀ alkyl, C₃-C₁₀ cycloalkyl, C₆-C₁₄ aryl, and C₅-C₁₄ heteroaryl, wherein the substituents may be unsubstituted or partially or completely fluorinated;
R² is selected from the group consisting of C₁-C₁₀ alkyl, C₃-C₁₀ cycloalkyl, C₆-C₁₄ aryl, and C₅-C₁₄ heteroaryl, wherein the substituents may be unsubstituted or partially or completely fluorinated;
R³ is a partially or completely fluorinated C₁-C₁₀ alkyl;
starting from a ketone of formula (II), 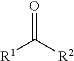
wherein:
R¹ is selected from the group consisting of C₁-C₁₀ alkyl, C₃-C₁₀ cycloalkyl, C₆-C₁₄ aryl, and C₅-C₁₄ heteroaryl, wherein the substituents may be unsubstituted or partially or completely fluorinated;
R² is selected from the group consisting of C₁-C₁₀ alkyl, C₃-C₁₀ cycloalkyl, C₆-C₁₄ aryl, and C₅-C₁₄ heteroaryl, wherein the substituents may be unsubstituted or partially or completely fluorinated;
and a carboxylic acid salt of formula (III) (R³COO)ₓY, wherein
R³ is a partially or completely fluorinated C₁-C₁₀ alkyl;
Y is a cation selected from the group consisting of K, Li, Na, Cs, Mg, Ca, Fe, Cu, Ag, and Zn;
x is 1 or 2; and
the R³ group of the carboxylic acid salt is transferred to the carbonyl carbon of the ketone of formula (II) with the release of CO₂.

2. The method according to claim 1, wherein the reaction is carried out in a solvent selected from the group consisting of DMF, NMP, DMAc, and DMSO.

3. The method according to claim 2, wherein DMF is the solvent.

4. The method according to claim 1, wherein the reaction temperature is in the range of 100° C. to 150° C.

5. The method according to claim 1, wherein the reaction temperature is in the range of 135° C. to 145° C.

6. The method according to claim 1, wherein the reaction is carried out under anhydrous conditions.

7. The method according to claim 1, wherein the reaction is carried out in the presence of a catalyst.

8. The method according to claim 7, wherein the catalyst is an iron catalyst.

9. The method according to claim 7, wherein the iron catalyst is selected from the group consisting of FeCl₂, FeCl₃, FeBr₃, FeF₃, FeTFA₃, FeSO₄, and Fe₂(SO₄)₃.

10. The method according to claim 9, wherein the quantity of iron catalyst ranges from 10 to 75 mol %, and preferably from 15 to 45 mol %.

11. The method according to claim 1, wherein R¹ is a C₁-C₁₀ alkyl, which may be unsubstituted or partially or completely fluorinated.

12. The method according to claim 1, wherein R³ is a C₁-C₁₀ alkyl, which may be unsubstituted or partially or completely fluorinated.

13. The method according to claim 1, wherein R¹ is selected from the group consisting of CH₃, CH₂F, CHF₂, CF₃, CH₂CH₃, CH₂CH₂F, CH₂CHF₂, CH₂CF₃, CF₂CF₃, CF(CF₃)₂, C(CF₃)₃, and CF₂CF₂CF₃.

14. The method according to claim 1, wherein R² is selected from the group consisting of CH₃, CH₂F, CHF₂, CF₃, CH₂CH₃, CH₂CH₂F, CH₂CHF₂, CH₂CF₃, CF₂CF₃, CF(CF₃)₂, C(CF₃)₃, and CF₂CF₂CF₃.

15. The method according to claim 1, wherein R³ is selected from the group consisting of CF₃, CF₂CF₃, CF(CF₃)₂, C(CF₃)₃, and CF₂CF₂CF₃.
